(19) United States
(12) Patent Application Publication
(10) Pub. No.: US 2022/0062507 A1
(43) Pub. Date: Mar. 3, 2022

(54) Nanostructured Bactericidal Polymer Foil

(71) Applicant: The Board of Trustees of the University of Illinois, Urbana, IL (US)

(72) Inventors: Qing Cao, Urbana, IL (US); Yi Zhang, Urbana, IL (US)

(21) Appl. No.: 17/460,561

(22) Filed: Aug. 30, 2021

Related U.S. Application Data
(60) Provisional application No. 63/072,231, filed on Aug. 31, 2020.

(51) Int. Cl. A61L 27/54; B05D 1/00; B05D 1/32; B05D 3/14; B05D 5/00; A61L 27/16; A61L 27/34

(52) U.S. Cl. CPC A61L 27/54; B05D 1/005; B05D 1/322; B05D 3/145; B05D 5/00; A61L 2300/404; A61L 27/34; A61L 2420/02; A61L 2400/12; A61L 2300/606; A61L 27/16

(57) ABSTRACT

Nanostructured thin films and methods of their manufacture are disclosed. An example method of manufacturing includes forming a colloidal crystal mask on a template substrate so as to partially mask the template substrate. The method additionally includes etching the exposed potions of the template substrate to form a plurality of holes within the template substrate corresponding to the plurality of holes in the first thin film. The method yet further includes depositing a second thin film on the etched template substrate to form a nanostructured thin film. The nanostructured thin film includes a nanopillar array disposed along a first surface of a flexible substrate. The flexible substrate and the nanopillar array comprise a polymer material.

### CROSS-REFERENCE TO RELATED APPLICATION

This application claims priority to U.S. provisional patent application No. 63/072,231, filed Aug. 31, 2020, which is hereby incorporated by reference in its entirety.

### STATEMENT REGARDING FEDERALLY SPONSORED RESEARCH OR DEVELOPMENT

This invention was made with government support under grant number 2015292 awarded by the National Science Foundation. The United States Government has certain rights in the invention.

### BACKGROUND

Biofouling, the accumulation of unwanted biological matter on surfaces, is a critical problem for a wide range of medical, marine, and industrial applications. For example, more than 45% of hospital-contracted infections are traced to biofilm-infected medical devices. As a first line of prevention, surface coating approaches capable of preventing bacterial adhesion and biofilm formation are highly desirable. Conventional antibacterial surfaces rely on the administration of toxic chemicals such as antibiotics. Their sustained use could lead to pathogenic resistance and toxic effects on human tissues or organs. The longevity of the coating effectiveness is also limited.

Recently, it has been discovered that some natural surfaces have physical bactericidal functionality. For example, cicada wings exhibit regular arrays of nanopillars with 50-100 nm diameter, ˜200 nm pitch, and ˜250 nm height, and are of high bactericidal efficacy achieved through mechanically rupturing the membranes of cells attached. However, reproduction of such high density, submicron, and high-aspect-ratio structures over large area pose fabrication challenges. In addition, the detailed bactericidal mechanism, especially the correlation between the geometrical parameters of the pillar arrays, their mechanical properties, and bactericidal efficacy, is still a pending question. These obstacles limit the capability to engineer the performance of biomimetic antimicrobial surfaces, and apply them in practical applications.

### SUMMARY

The embodiments described herein provide nanostructured thin films and methods of their manufacture to provide a non-toxic bacteria-killing surface.

In a first aspect, a nanostructured thin film may be provided. The nanostructured thin film may include a nanopillar array disposed along a first surface of a flexible substrate. The nanopillar array includes a plurality of nanopillars. The plurality of nanopillars are disposed according to a pitch in the range of 100-500 nm. Each nanopillar of the plurality of nanopillars comprises a diameter in the range of 50-250 nm and a height in the range of 200-1000 nm.

In a second aspect, a method of manufacturing a nanostructured thin film is provided. The method includes forming a colloidal crystal mask on a template substrate. The method also includes controllably etching the colloidal crystal mask so as to provide a desired gap between adjacent elements of the colloidal crystal mask. The method further includes depositing a first thin film on the template substrate and the etched colloidal crystal mask such that at least a portion of the first thin film is formed along the template substrate within the desired gap between adjacent elements of the colloidal crystal mask. The method yet further includes removing the colloidal crystal mask leaving a plurality of holes in the first thin film corresponding to the adjacent elements of the colloidal crystal mask, the plurality of holes in the first thin film exposing the template substrate. The method also includes anisotropically etching the exposed potions of the template substrate to form a plurality of vertical holes within the template substrate corresponding to the plurality of holes in the first thin film. The method also includes depositing a second thin film on the etched template substrate to form a nanostructured thin film. The nanostructured thin film includes a nanopillar array disposed along a first surface of a flexible substrate. The flexible substrate and the nanopillar array include a polymer material.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

Example embodiments should become apparent from the following description, which is given by way of example only, of at least one preferred but non-limiting embodiment, described in connection with the accompanying figures.

FIG. 1A illustrates nanostructured cicada wings with physical bactericidal capability.

FIG. 1B illustrates a physical bactericidal mechanism.

FIG. 2 illustrates a smart coating on an implant with antimicrobial and structural-health monitoring dual functionalities, according to an example embodiment.

FIG. 3 illustrates a nanostructured thin film, according to an example embodiment.

FIG. 4 illustrates SEM images of polymer pillar arrays with varied pillar heights and their corresponding bactericidal efficacy against E. coli, according to example embodiments.

FIG. 5 illustrates SEM images of nanopillar arrays with varied characteristics, according to example embodiments.

FIG. 6 illustrates normalized counts of E. coli and S. aureus in solution after incubation with the nanopillar arrays for 90 min as a function of nanopillar height and pitch, according to example embodiments.

FIG. 7 illustrates counts of S. aureus and P. aeruginosa in solution after incubation for 30 min and their accumulation on surface as biofilms after incubation for 24 hours for planar control and nanopillar arrays, according to example embodiments.

FIG. 8 illustrates a method, according to example embodiments.

FIG. 9 illustrates a method of fabricating nanopillar arrays on polymer thin films, according to an example embodiment.

FIG. 10 illustrates SEM images of polymer nanopillars after soaking and drying cycle, according to example embodiments.

FIG. 11 illustrates relevant mammalian cell viability on biomimetic antimicrobial nanopillar arrays, according to example embodiments.

FIG. 12 illustrates optical images of the skin tissues around planar control and the nano-structured antimicrobial polymer film inoculated with P. aeruginosa and normalized counts of S. aureus and P. aeruginosa, and the count of the collected live bacteria (CFU: colony forming unit) according to example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein. Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Overview

Embodiments of the present disclosure include high density nanopillar arrays and methods for preparing high density nanopillar arrays. Certain embodiments include a method of preparing high density nanopillar arrays with independently adjustable pillar height, radius, and spacing, on various polymer substrates with a wide range of Young's moduli. With optimized geometries, these nanostructures can effectively kill attached bacteria through rupturing their cell membranes in a purely mechanical stretching process, and thus offer a cost-effective, “chemical-free” and wide-spectrum strategy to prevent bacteria-related infections and fouling.

In various embodiments, devices could include a large-area flexible polymer film. The polymer film could include a first side that includes a well-controlled nanopillar array structure fabricated using nanosphere lithography, highly anisotropic silicon deep etching, and micro-molding techniques. In some examples, the polymer film could be produced according to a master template. The master template could be formed from a silicon (Si) substrate with a silicon dioxide (SiO₂) layer on top based on a low-cost wafer-scale nanosphere lithography process. In some examples, the master template could be formed using a colloidal crystal mask made of mono-dispersed polystyrene nanospheres distributed along the SiO₂ layer. An oxygen-based reactive-ion etch (RIE) process could be utilized to reduce the sphere size to create controllable gaps between neighboring spheres. Once the appropriate gap is obtained by etching, a thin metal film could be deposited. The colloidal crystal mask may then be removed (e.g., by a liftoff technique). The remaining SiO₂ layer could be etched using a CHF₃ RIE process, the metal thin film could be removed using appropriate wet etchant, and the exposed Si surface could be etched using a deep RIE process with the SiO₂ pattern as the hard mask.

In some examples, the present disclosure describes processes for fabricating a nanostructured, anti-microbial surface on a polymer substrate. Embodiments of the present disclosure include a cost-effective way to make polymer thin films with precisely-controlled submicron features over large area. In certain embodiments, the engineered bactericidal films are further utilized as substrates for flexible electronics systems that may, for example, be integrated on the surface of biomedical implants for prolonged antibacterial and sensing functionalities. Embodiments may help to reduce risks associated with infection, monitor the structural health of the implant, and measure the physiological conditions of the surrounding tissues.

Embodiments of the present disclosure can be used in various applications to reduce bacteria growth on the surface (e.g., food packaging, protecting public surfaces, reducing nosocomial (hospital-acquired) infections for patients, etc. Further, certain embodiments may also serve as a functional substrate for wearable or implantable electronics with mechanically flexible form factor.

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

All of the patents, patent applications, patent application publications and other publications recited herein are hereby incorporated by reference as if set forth in their entirety.

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the scope of the invention as set forth in the claims.

### II. Example Nanostructured Thin Films

“Biomimicry”, which combines the words “bios” (life) and “mimesis” (to imitate), is a relatively new scientific field focused on the study of nature's ability to adapt to a diverse range of environmental conditions. One of the results of biomimicry has been the realization of nanoengineered surfaces (NES) that replicate nature's adaptation to: attract or repel various liquids, adhere to or release from surfaces in wet or dry conditions, endure wear, resist hostile corrosive environments, manage heat transfer, and/or manage incident light.

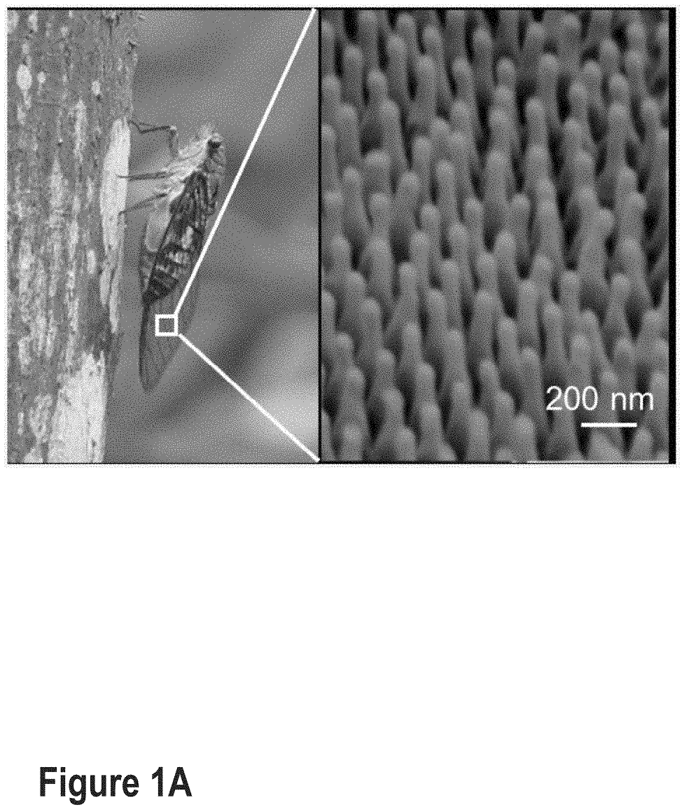

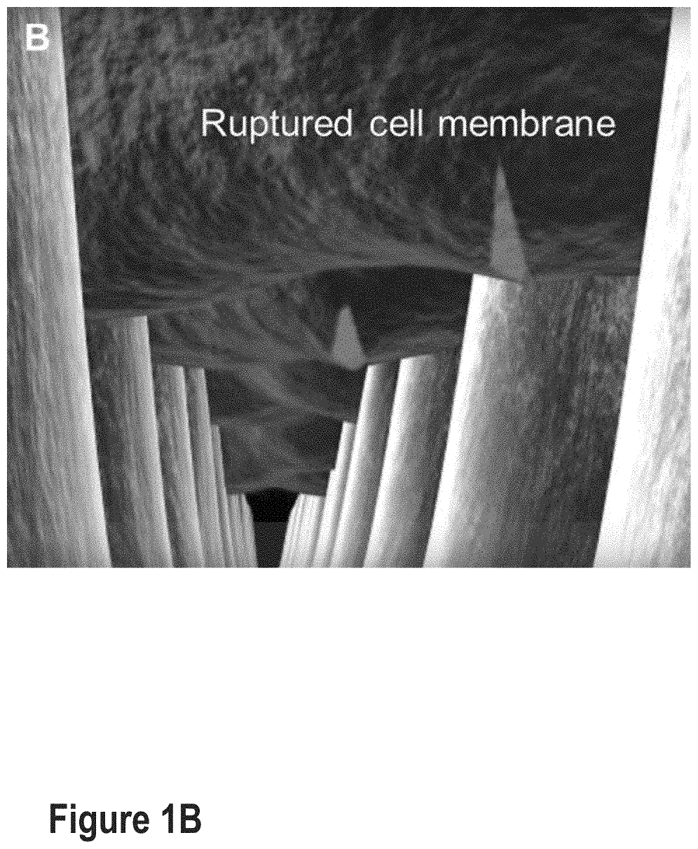

For example, the wings of the cicada exhibit regular arrays of nanopillars that have high bactericidal efficacy by mechanically rupturing the membranes of cells that attach to wing surfaces. FIG. 1A illustrates nanostructured cicada wings with physical bactericidal capability. Arrays of nanopillars with 50-100 nm diameter, ˜200 nm pitch, and 200-400 nm height cover the surfaces of cicada wings and render them capable of killing attached bacteria through purely physical interactions without release of chemicals.

FIG. 1B illustrates a possible related physical bactericidal mechanism. It is speculated that when bacteria contact the nanopillar arrays, regions of the microorganisms are suspended between the pillars since bacteria are typically 5-50 times larger than the pillar pitch. Stretching occurs in these suspended regions, rupturing cell membranes of bacteria and causing cell necrosis. Observing this property has motivated researchers to attempt reproduction of this naturally-occurring surface.

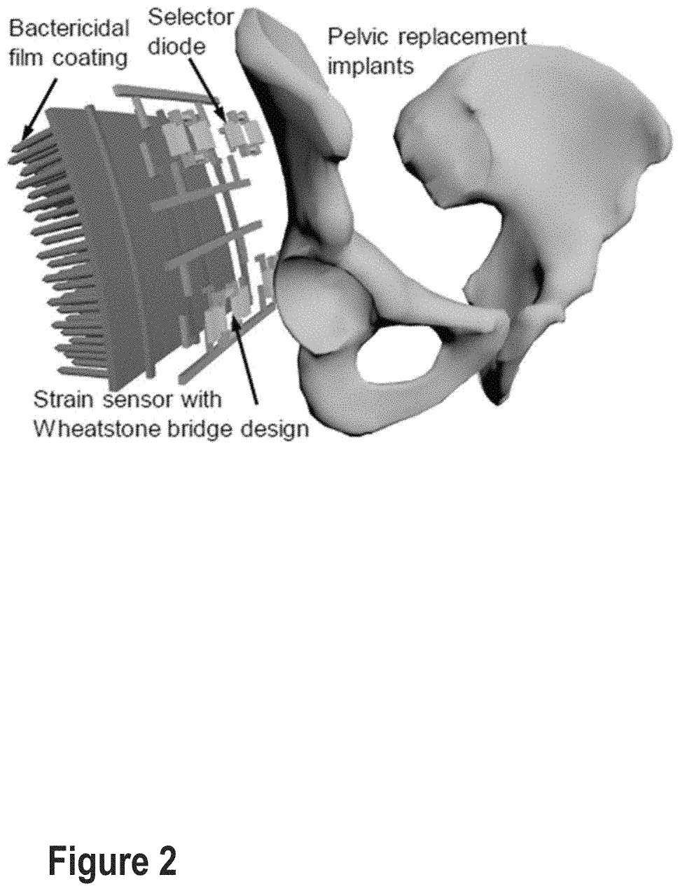

As one example, nanostructured thin films described herein could be applied to surfaces of an implantable medical device to reduce infection. FIG. 2 illustrates a smart coating on an orthopedic implant with antimicrobial and structural-health monitoring dual functionalities, according to an example embodiment. It will be understood that antimicrobial thin films may be beneficially utilized in many applications, including but not limited to those in healthcare, medical research, and/or public health fields.

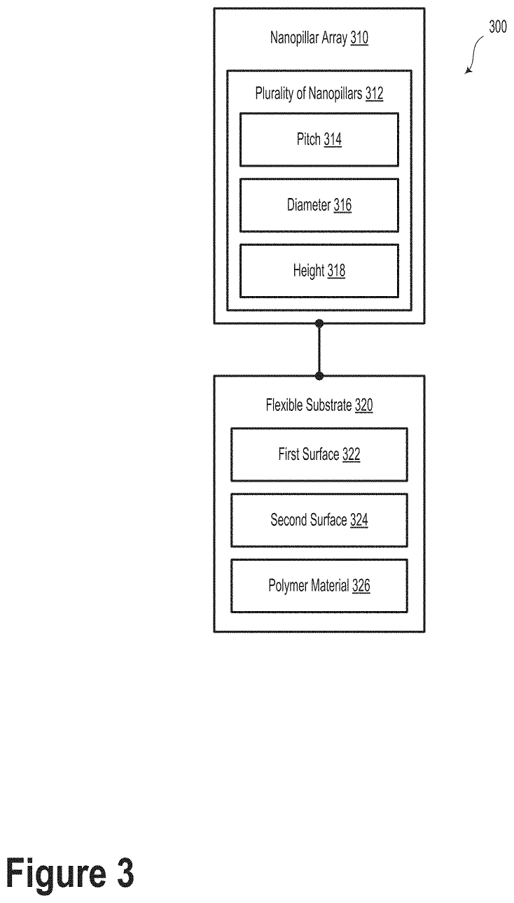

FIG. 3 illustrates a nanostructured thin film 300, according to an example embodiment. The nanostructured thin film 300 includes a nanopillar array 310 disposed along a first surface 322 of a flexible substrate 320. The nanopillar array 310 includes a plurality of nanopillars 312. The plurality of nanopillars 312 are disposed according to a pitch 314 in the range of 100-500 nm. In such a scenario, each nanopillar of the plurality of nanopillars 312 may include a diameter 316 in the range of 50-200 nm and/or a height 318 in the range of 200-1000 nm.

It will be understood that other dimensions of the nanopillars and/or nanopillar array 310 are possible and contemplated. For example, the pitch 314 could be about 200 nm. Additionally or alternatively, the pitch 314 could be about 220 nm and the diameter 316 could be about 100 nm, and the height 318 could be about 400 nm. Additionally or alternatively, the pitch 314 could be about 200 nm, the diameter 316 could be in the range of 50-100 nm, and the height 318 could be about 250 nm.

In some embodiments, the nanopillar array 310 is configured to provide a non-toxic bacteria-killing surface, as described herein. In some examples, the nanopillar array 310 could include an area of at least 3 inches by 3 inches. It will be understood that nanopillar arrays with even larger areas are possible and contemplated. For example, by utilizing roll-to-roll fabrication techniques, stamping, or other methods, very large areas of nanopillars may be realized.

In some embodiments, the flexible substrate 320 and the plurality of nanopillars 312 could include a polymer material 326. For example, the polymer material 326 could include at least one of: polyimide, polymethylmethacrylate, polydimethylsiloxane, polyethylene, polypropylene, polybutadiene, polyisoprene, polychloroprene, polystyrene, polyvinyl chloride, vinyl acetate, polyurethane, silicone, or polytetrafluoroethylene. It will be understood that other polymer materials are possible and contemplated.

The mechanical stiffness of the polymer nanopillars can also be adjusted over a wide range through either modifying the degree of cross-link in the polymer curing process or adopting different polymer materials, from polydimethylsiloxane (modulus ˜2.5 MPa), polyurethane (modulus 20-320 MPa), polypropylene (modulus ˜1.3 GPa), and to various polyimide (modulus 2.5-10 GPa), among other possibilities.

In various examples, the flexible substrate 320 could be is configured to be coupled, by way of a second surface 324 of the flexible substrate 320, to at least one of: a flexible electronic device, a wearable electronic device, an implantable electronic device, an implantable medical device, a non-implantable medical device, or a high-touch surface.

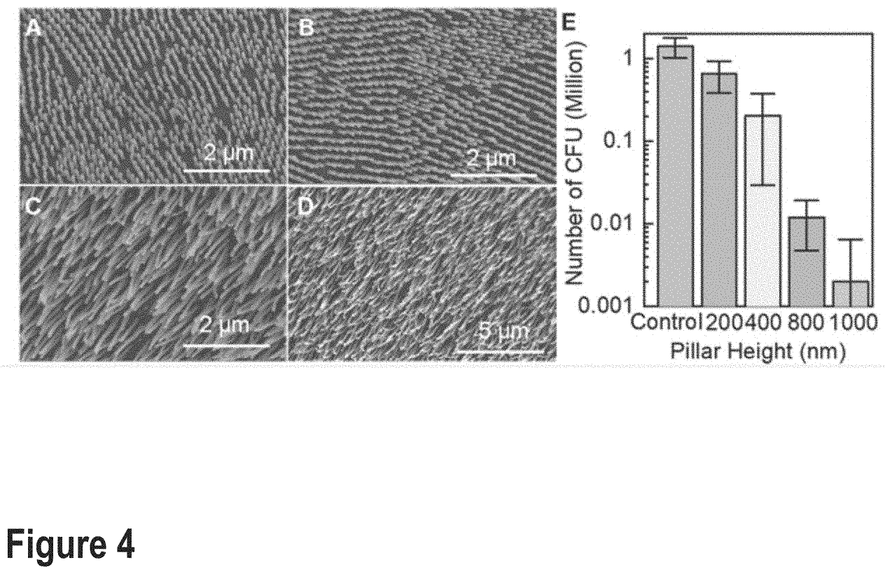

FIG. 4 illustrates SEM images of polymer pillar arrays with varied pillar heights, according to example embodiments. FIG. 4 includes tilted-view SEM micrographs of polymer nanopillar arrays with varied nanopillar heights (A: 200 nm; B: 400 nm; C: 800 nm; D: 1 μm). E: The number of colony forming units (CFUs) in the suspensions after 90 min incubation as an illustration of the bactericidal efficacy. In such examples, the control was a planar polymer foil.

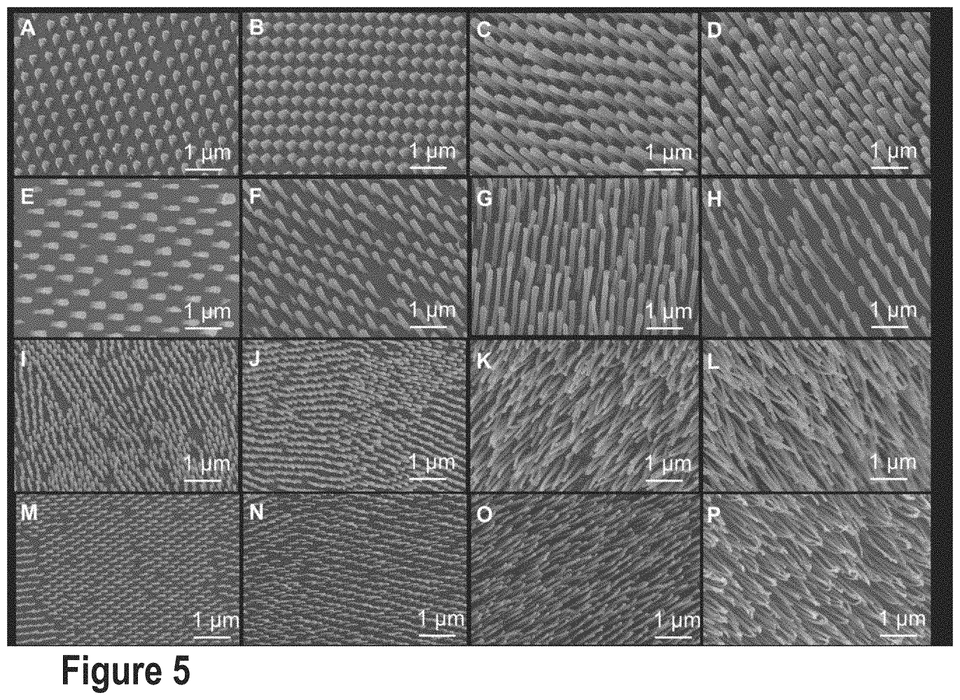

FIG. 5 illustrates SEM images of nanopillar arrays with varied characteristics, according to example embodiments. The SEM images of FIG. 5 include oblique (tilted) angle images of nanopillars with (A-D) 250 nm diameter, 500 nm pitch nanopillar arrays with the pillar heights varied from 200 nm (frame A), 400 nm (frame B), 800 nm (frame C), to 1 μm (frame D). (E-H) 100 nm diameter, 500 nm pitch nanopillar arrays with the pillar heights varied from 200 nm (frame E), 400 nm (frame F), 800 nm (frame G), to 1 μm (frame H). (I-L) 100 nm diameter, 240 nm pitch nanopillar arrays with the pillar heights varied from 200 nm (frame I), 400 nm (frame J), 800 nm (frame K), to 1 μm (frame L). (M-P) 50 nm diameter, 240 nm pitch nanopillar arrays with the pillar heights varied from 200 nm (frame M), 400 nm (frame N), 800 nm (frame 0), to 1 μm (frame P).

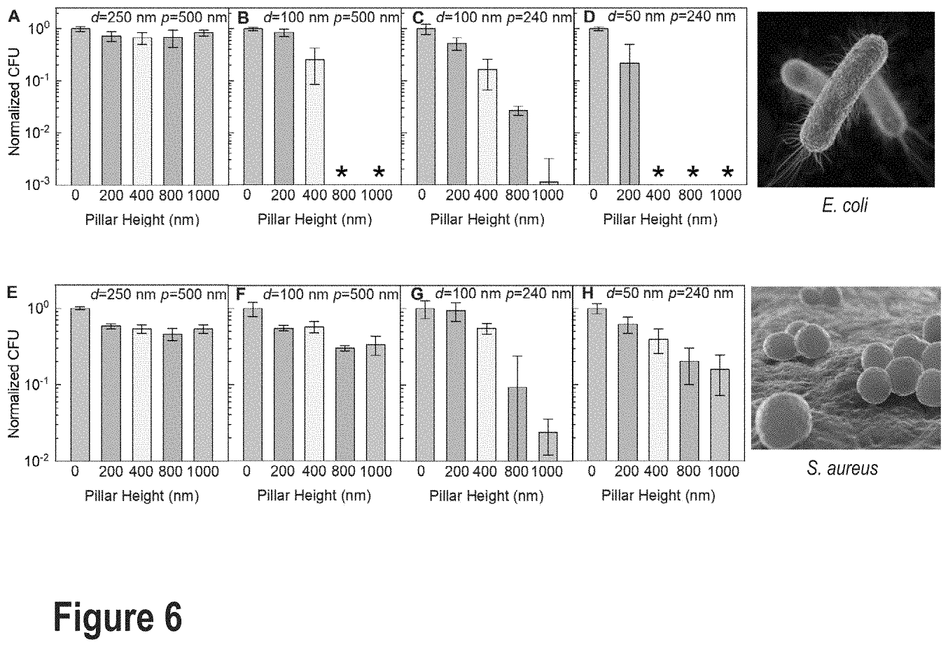

FIG. 6 illustrates normalized counts of E. coli and S. aureus in solution as a function of nanopillar height and pitch, according to example embodiments. About 1.5 million Escherichia coli (strain M1655) or methicillin-resistant Staphylococcus aureus (USA 300) suspended in phosphate-buffered saline (PBS) solution were incubated on nanostructured polymer films, together with the planar controls, for 30 min (E. coli) or 90 min (S. aureus). The cell suspensions were then collected with sufficient washing, further diluted, and spread on nutrient agar plates to determine the number of viable bacteria in term of the colony forming unit (CFUs).

FIG. 6 illustrates (A-D) Normalized counts of viable E. coli (MG1655) remaining in suspension after incubated with 1×1 cm² nanopillar arrays with varying geometries for 30 min. * indicates that no colony forming unit (CFU) was identified in all three samples tested. (E-H) Normalized counts of viable methicillin-resistant S. aureus (USA300) remaining in suspension after incubated with 1×1 cm² nanopillar arrays with varying geometries for 90 min. Frame A and E: 500 nm pitch, 250 nm diameter pillar arrays; Frame B and F: 500 nm pitch, 100 nm diameter pillar arrays; Frame C and G: 240 nm pitch, 100 nm diameter pillar arrays; Frame D and H: 240 nm pitch, 50 nm diameter pillar arrays. For each combination of pillar pitch and diameter, the pillar heights were varied from 200 nm to 1 Zero pillar height indicates the planar control.

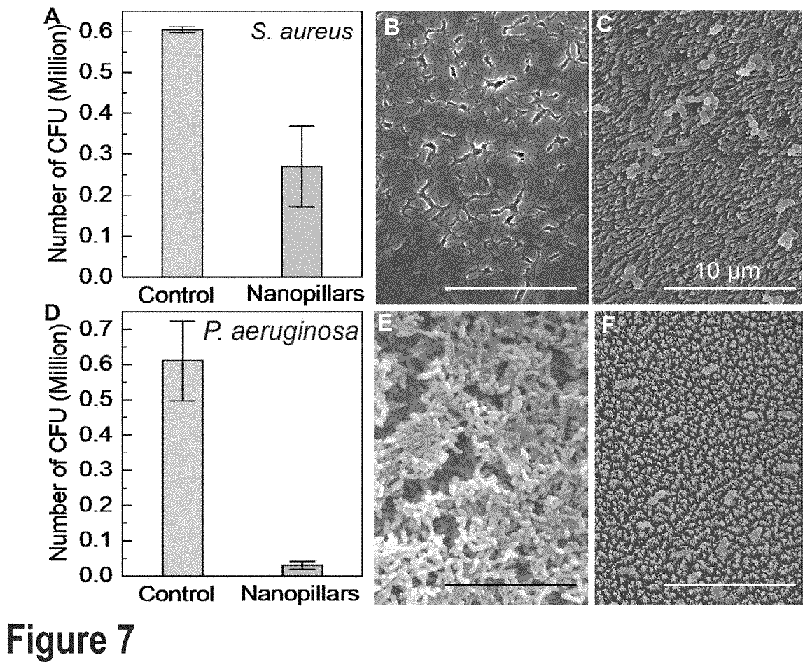

FIG. 7 illustrates counts of viable S. aureus and P. aeruginosa after incubation and biofilm formation on planar control and nanopillar arrays, according to example embodiments. (A) S. aureus cell viability after incubation with planar control and nanopillar arrays. S. aureus biofilms formed on (B) planar control and (C) polymer nanopillar arrays after 24 hr. (D) P. aeruginosa cell viability after incubation with planar control and nanopillar arrays. P. aeruginosa biofilms formed on (E) control and (F) polymer nanopillar arrays after 24 hours.

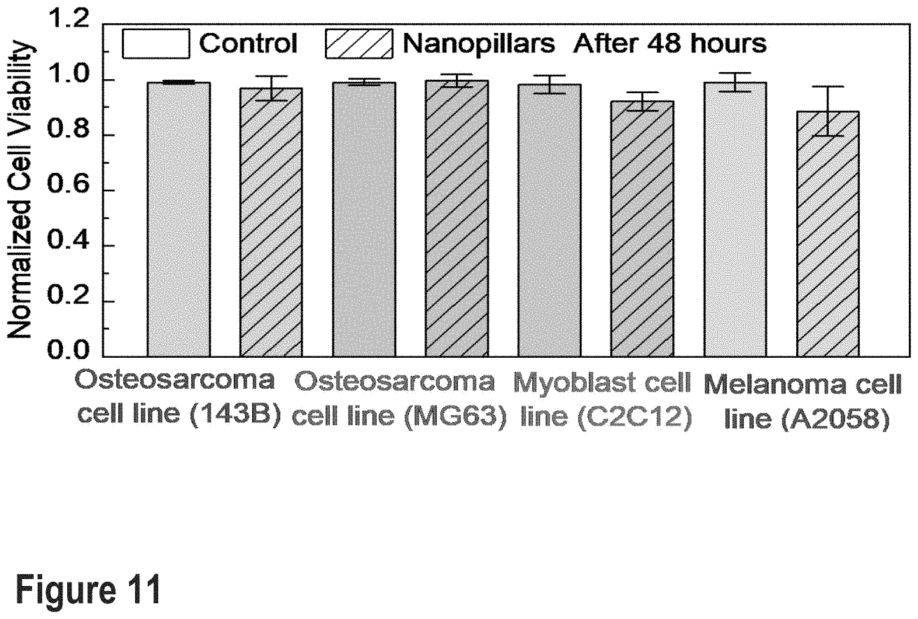

In example embodiments, the nanopillar arrays described herein may be configured to have a bactericidal effect on both gram-positive bacteria and gram-negative bacteria. Based on the experimental bactericidal results, the formed polyimide nano-pillar arrays can effectively kill both gram-positive (S. aureus) and gram-negative (P. aeruginosa) bacteria, without affecting human osteosarcoma cells, melanoma cells, and mice myoblast cells, with the cell viability determined by the standard plate-count technique as shown in FIG. 11. Moreover, for the same bacteria of E. coli, the quantitative bactericidal efficacy, measured as the number of bacteria killed by these polymer nanopillars after 90 min incubation, heavily depends on the pillar array topology, as exemplified for the pillar height, pitch, and diameter variations (FIG. 5 and FIG. 6).

### III. Example Methods

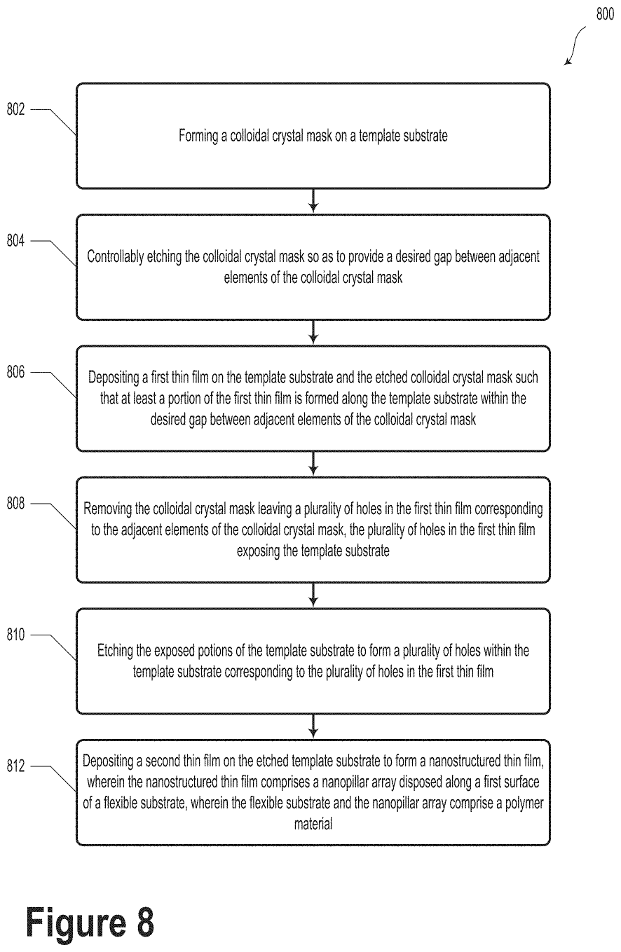

FIG. 8 illustrates a method 800 of manufacturing a nanostructured thin film, according to example embodiments. While method 800 illustrates several blocks of a method, it will be understood that fewer or more blocks or steps could be included. In such scenarios, at least some of the various blocks or steps may be carried out in a different order than of that presented herein. Furthermore, blocks or steps may be added, subtracted, transposed, and/or repeated.

Block 802 includes forming a colloidal crystal mask on a template substrate. In some examples, the template substrate could include an SiO₂/Si wafer.

Block 804 includes controllably etching the colloidal crystal mask so as to provide a desired gap between adjacent elements of the colloidal crystal mask. In some examples, controllably etching the colloidal crystal mask could include etching the colloidal crystal mask by an oxygen reactive-ion etch (RIE).

Block 806 includes depositing a first thin film on the template substrate and the etched colloidal crystal mask such that at least a portion of the first thin film is formed along the template substrate within the desired gap between adjacent elements of the colloidal crystal mask. In some examples, the first thin film could include a metal material.

Block 808 removing the colloidal crystal mask leaving a plurality of holes in the first thin film corresponding to the adjacent elements of the colloidal crystal mask, the plurality of holes in the first thin film exposing the template substrate.

Block 810 includes etching the exposed potions of the template substrate to form a plurality of holes within the template substrate corresponding to the plurality of holes in the first thin film. In the case of the template substrate including an SiO₂/Si wafer, etching the exposed portions of the template substrate could include 1) etching the exposed SiO₂ of the template substrate by CHF₃ RIE; and 2) etching the underlying Si of the template substrate by a deep Si RIE process.

Block 812 includes depositing a second thin film on the etched template substrate to form a nanostructured thin film (e.g., nanostructured thin film 300). The nanostructured thin film includes a nanopillar array disposed along a first surface of a flexible substrate. The flexible substrate and the nanopillar array include a polymer material. In some examples, forming the second thin film on the etched template substrate could include coating precursors/solutions for polymers with different moduli, and curing the precursors/solutions.

In some examples, the polymer material could include at least one of: polyimide, polymethylmethacrylate, polydimethylsiloxane, polyethylene, polypropylene, polybutadiene, polyisoprene, polychloroprene, polystyrene, polyvinyl chloride, vinyl acetate, polyurethane, silicone, or polytetrafluoroethylene.

In some embodiments, the method 800 also includes releasing the nanostructured thin film from the etched template substrate so as to provide a non-toxic bacteria-killing surface.

In various examples, forming the colloidal crystal mask on the template substrate could include depositing a monolayer of colloidal crystals on the template substrate by way of at least one of: a spin coating technique or a Langmuir-Blodgett (LB) technique.

Additionally or alternatively, the colloidal crystal mask could include mono-dispersed polystyrene nanospheres.

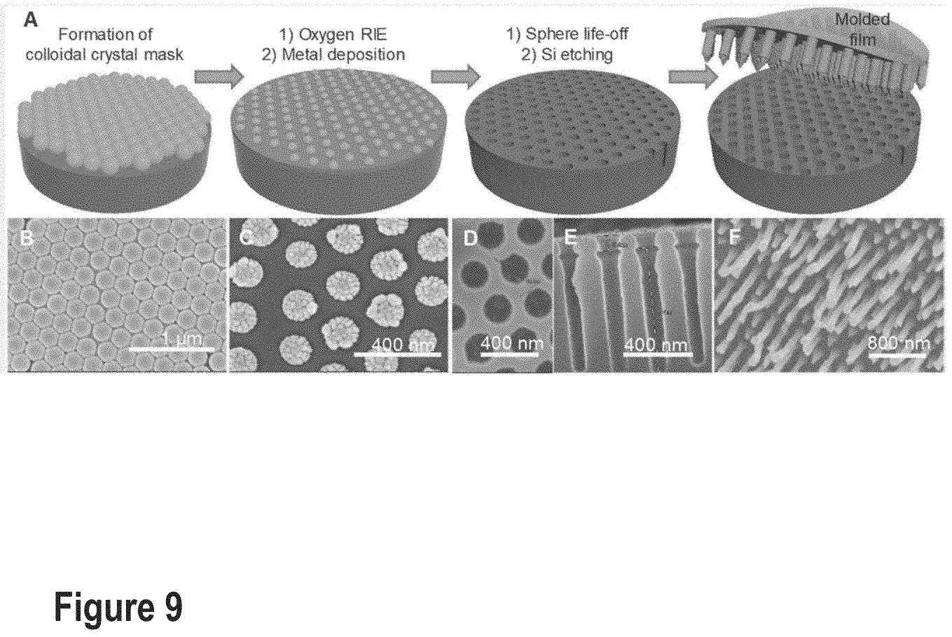

FIG. 9 illustrates a method of fabricating nanopillar arrays on polymer thin films, according to an example embodiment. FIG. 9 illustrates a method of forming a large-area flexible controlled nanopillar array structure in accordance with an embodiment of the present disclosure. As an example, the nanopillar array structure could be fabricated through a molding process.

FIG. 9 illustrates (A) a schematic process flow; (B) Top-view SEM of the colloidal crystal mask before O₂ RIE; (C) Top-view SEM of the colloidal crystal mask after O₂ RIE; (D) the metal hard mask formed after lift-off; (E) cross-sectional SEM of the deep etched Si/SiO₂ substrate; and (F) nanopillar-array structures formed on Kapton film.

In such scenarios, a master template is created on a SiO₂/Si wafer using the low-cost wafer-scale nanosphere lithography process. Here, colloidal crystal mask made of mono-dispersed polystyrene nanospheres is deposited across the whole wafer. An oxygen reactive-ion etching (RIE) trims down the sphere size (e.g., sphere diameter) to create controllable gaps between neighboring spheres, which are subsequently covered by a deposited metal thin film. The colloidal crystal mask is then removed, followed by the etching of the exposed SiO₂ by CHF₃ RIE and the underlying Si by deep Si RIE in sequence. Precursors/solutions for polymers with different moduli are coated onto the template, and thermally cured to form thin films with the complementary nanopillar array structure. The pillar pitch, diameter, and height are controlled by the nanosphere size, the oxygen ME and the deep Si RIE time, respectively. The bactericidal efficacy of films with systematically varied nanopillar geometrical parameters and Young's modulus are characterized in experiment. The design flexibility, in term of both geometries and material properties, allows for fabrication of artificial surfaces that can outperform the results of natural evolution.

In example embodiments, the starting template substrate may include a single-crystalline Si wafer covered by a thin layer of oxide as substrate. Mono-dispersed polystyrene nanospheres are deposited on the template substrate by spin casting. Upon drying, nanospheres self-assemble into a hexagonal-close-packed monolayer. A timed oxygen-plasma RIE may then be utilized to trim down the sphere size and create controllable inter-sphere gaps, which will be covered by subsequently deposited 10 nm-thick Cr thin film. After metal deposition, the colloidal crystal mask is removed in chloroform with applied sonication, followed by etching of the exposed SiO₂ by CF₄ RIE, with the Cr film as the etching mask. Next, the Cr layer is removed by wet etching to make the sample ready for deep-Si RIE, which will finally create the high-aspect-ratio nanowells using the patterned SiO₂ as the hard etching mask. In our process, the nanowell pitch, diameter, and depth are separately and precisely controlled by the starting polystyrene nanosphere size, the etching time of the oxygen-plasma RIE, and the number of etching cycles in deep-Si RIE, respectively. After obtaining master templates, polyamic acid dissolved in N-methyl-2-pyrrolidinone (NMP) was coated onto the master template using doctor blading. Vacuum annealing converted the oligomers into cross-linked polyimide that can be subsequently peeled off from the template as a standalone film, featuring high-density nanopillar arrays on its surface.

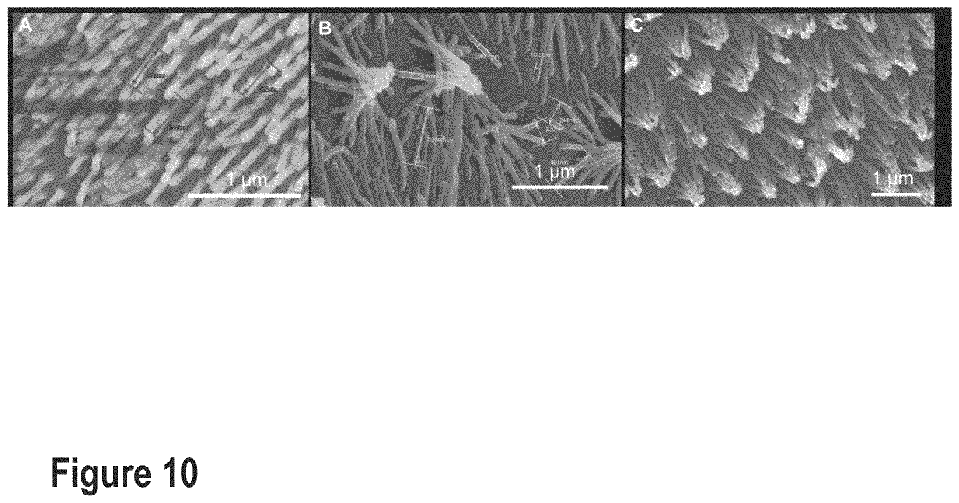

FIG. 10 illustrates SEM images of polymer nanopillars, according to example embodiments. FIG. 10 includes tilted-view SEM micrographs of polymer nanopillar arrays after being exposed to buffer solution and air dried. (A) 100 nm diameter, 240 nm pitch, 400 nm tall pillar arrays. (B) 50 nm diameter, 240 nm pitch, 400 nm tall pillar arrays. (C) 100 nm diameter, 240 nm pitch, 800 nm tall pillar arrays. It indicates that an optimized pillar geometry needs to be selected in order to ensure both high bactericidal efficacy and structural stability.

FIG. 11 illustrates relevant mammalian cell viability on biomimetic antimicrobial nanopillar arrays, according to example embodiments. In an experiment, the films were incubated with 1 mg/ml Neomycin for 12 hours at 37° C., and then washing with PBS solution 3 times. While drying the film, UV sterilization was performed for 30 min. Then, 1.5×10⁶ of various types of mammalian cells were seeded onto the films and incubated for 48 hours. Cell cytotoxicity was examined by WST-1 assay, and no sign of cytotoxicity was observed.

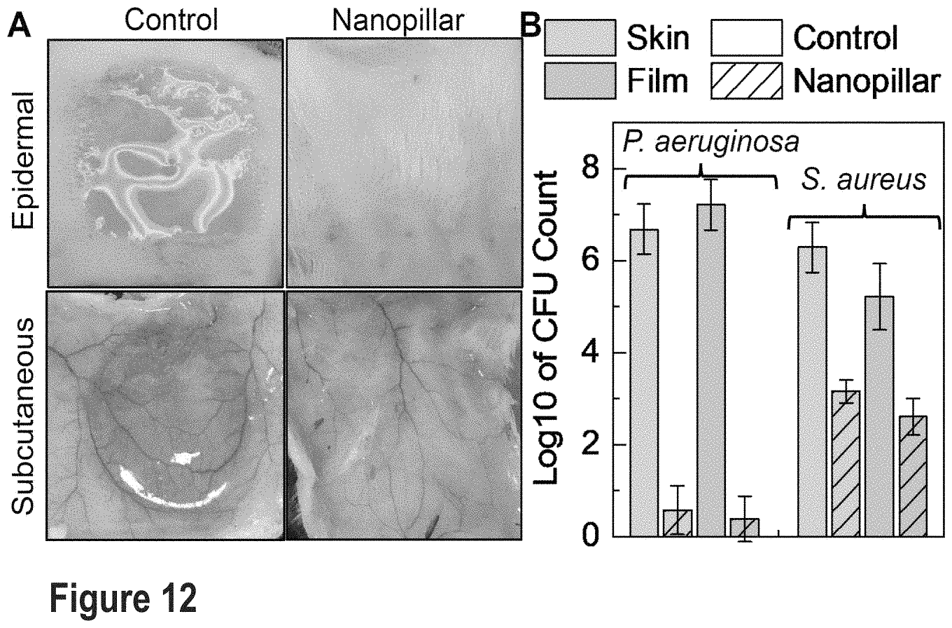

FIG. 12 illustrates optical images of the skin tissues around planar control and the nano-structured antimicrobial polymer film inoculated with five million CFUs of S. aureus and P. aeruginosa, according to example embodiments. FIG. 12 illustrates (A) Optical images of the skin tissues around planar control and the nano-structured antimicrobial polymer film inoculated with P. aeruginosa after three days. (B) Comparison of the number of CFUs.

The biomimetic properties of nanopillar arrays to prevent infection in vivo has been confirmed in a modified tape-stripping infection model of mice. Six-to nine-week-old CD-1 mice (cohorts of 6, equal ratio of males and females) were anesthetized by intraperitoneal injection of ketamine (80-100 mg/kg) and xylazine (10-12.5 mg/kg). The fur on the dorsal of mice was removed by shaving followed by exfoliating cream. Then, an area of ca. 2 cm² was tape stripped with Tensoplast, an elastic adhesive bandage, 10 times in succession to disrupt the skin barrier by partial removal of the epidermal layer. The nanostructured physical antimicrobial films, along with planar controls, which had been sterilized and incubated for 12 hours at 37° C. with 1) 5×10⁶ CFUs of S. aureus strain ATCC 29213, or 2) 5×10⁶ CFUs of P. aeruginosa strain 27853105, in their liquid suspensions to mimic the surgical-site attachment of bacteria on implants, were affixed onto the tape-stripped skin with surgical tape. Infected mice were monitored for 3 days, and euthanized by over-dosing with CO₂. Compared to the planar controls, the films with biomimetic antimicrobial nanopillar arrays effectively prevent superficial infection as evident from the absence of pus and hemorrhage. The polymer films and the surrounding skin tissues were collected enbloc from all animals and vortexed for 1 min in 1 mL sterile saline to collect supernatant for CFU counting. The number of CFUs on the nanopillar films and skin in contact was many orders of magnitude, i.e. at least 1,000×, lower than that of the control samples.

### IV. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A nanostructured thin film, comprising:
a nanopillar array disposed along a first surface of a flexible substrate, wherein the nanopillar array comprises a plurality of nanopillars, wherein the plurality of nanopillars are disposed according to a pitch in the range of 100-500 nm, wherein each nanopillar of the plurality of nanopillars comprises a diameter in the range of 50-250 nm and a height in the range of 200-1000 nm.

2. The nanostructured thin film of claim 1, wherein the nanopillar array is configured to provide a non-toxic bacteria-killing surface.

3. The nanostructured thin film of claim 1, wherein the flexible substrate and the plurality of nanopillars comprise a polymer material.

4. The nanostructured thin film of claim 3, wherein the polymer material comprises at least one of: polyimide, polymethylmethacrylate, polydimethylsiloxane, polyethylene, polypropylene, polybutadiene, polyisoprene, polychloroprene, polystyrene, polyvinyl chloride, vinyl acetate, polyurethane, silicone, or polytetrafluoroethylene.

5. The nanostructured thin film of claim 1, wherein the pitch is about 200 nm.

6. The nanostructured thin film of claim 1, wherein the pitch is about 220 nm, the diameter is about 100 nm, and the height is about 400 nm.

7. The nanostructured thin film of claim 1, wherein the pitch is about 200 nm, the diameter is in the range of 50-100 nm, and the height is about 250 nm.

8. The nanostructured thin film of claim 1, wherein the flexible substrate is configured to be coupled to at least one of: a flexible electronic device, a wearable electronic device, an implantable electronic device, an implantable medical device, a non-implantable medical device, or a high-touch surface.

9. The nanostructured thin film of claim 1, wherein the nanopillar array is configured to have a bactericidal effect on both gram-positive bacteria and gram-negative bacteria.

10. A method of manufacturing a nanostructured thin film, comprising:
forming a colloidal crystal mask on a template substrate;
controllably etching the colloidal crystal mask so as to provide a desired gap between adjacent elements of the colloidal crystal mask;
depositing a first thin film on the template substrate and the etched colloidal crystal mask such that at least a portion of the first thin film is formed along the template substrate within the desired gap between adjacent elements of the colloidal crystal mask;
removing the colloidal crystal mask leaving a plurality of holes in the first thin film corresponding to the adjacent elements of the colloidal crystal mask, the plurality of holes in the first thin film exposing the template substrate;
etching the exposed potions of the template substrate to form a plurality of holes within the template substrate corresponding to the plurality of holes in the first thin film; and
depositing a second thin film on the etched template substrate to form a nanostructured thin film, wherein the nanostructured thin film comprises a nanopillar array disposed along a first surface of a flexible substrate, wherein the flexible substrate and the nanopillar array comprise a polymer material.

11. The method of claim 10, further comprising:
releasing the nanostructured thin film from the etched template substrate so as to provide a non-toxic bacteria-killing surface.

12. The method of claim 10, wherein forming a colloidal crystal mask on the template substrate comprises depositing a monolayer of colloidal crystals on the template substrate by way of at least one of: a spin coating technique or a Langmuir-Blodgett (LB) technique.

13. The method of claim 10, wherein the colloidal crystal mask comprises mono-dispersed polystyrene nanospheres.

14. The method of claim 10, wherein controllably etching the colloidal crystal mask comprises etching the colloidal crystal mask by an oxygen reactive-ion etch (ME).

15. The method of claim 10, wherein the template substrate comprises an SiO₂/Si wafer.

16. The method of claim 15, wherein etching the exposed portions of the template substrate comprises:
etching the exposed SiO₂ of the template substrate by CHF₃ RIE; and
etching the underlying Si of the template substrate by a deep Si RIE process.

17. The method of claim 10, wherein forming the second thin film on the etched template substrate comprises coating precursors/solutions for polymers with different moduli, and curing the precursors/solutions.

18. The method of claim 10, wherein the polymer material comprises at least one of: polyimide, polymethylmethacrylate, polydimethylsiloxane, polyethylene, polypropylene, polybutadiene, polyisoprene, polychloroprene, polystyrene, polyvinyl chloride, vinyl acetate, polyurethane, silicone, or polytetrafluoroethylene.

19. The method of claim 10, wherein the first thin film comprises a metal material.

20. The method of claim 10, wherein the pitch of the nanopillar array and the diameter and the height of the plurality of nanopillars are controlled by the sphere size, the oxygen RIE and the deep Si RIE time, respectively.
